(19) United States
(12) Patent Application Publication
(10) Pub. No.: US 2008/0113046 A1
(43) Pub. Date: May 15, 2008

(54) Use of Omega-3 Fatty Acid(s) for Treating Hypercholesterolemia Caused by Anti-Retroviral Treatment of Hiv-Infected Patients

(75) Inventor: Jean Gardette, Neuilly Sur Seine (FR)

Correspondence Address: BIRCH STEWART KOLASCH & BIRCH, PO BOX 747, FALLS CHURCH, VA, 22040-0747, US

(73) Assignee: Pierre Fabre Medicament, Boulogne-billancourt (FR)

(21) Appl. No.: 11/792,060

(22) PCT Filed: Dec. 2, 2005
(86) PCT No.: PCT/EP05/56431

§ 371 Date: Jun. 26, 2007

(30) Foreign Application Priority Data
Dec. 3, 2004 (FR) 0412862

(51) Int. Cl. A61K 31/202; A61K 31/225; A61K 36/55; A61K 36/42

(52) U.S. Cl. 424/758; 514/560; 514/547; 424/768; 424/776

(57) ABSTRACT

The invention relates to the use of omega-3 fatty acid(s) or the marine and/or vegetable omega-3 fatty acid(s) source for preparing a drug for treating a hypercholesterolemia caused by the anti-retroviral treatment of patients infected by human immunodeficiency virus (HIV) by limiting a non-HDL fraction of circulating lipoproteins and to a combined product comprising at least one type of omega-3 fatty acid and at least one type of anti-retroviral agent for simultaneous, separate or temporally gradual use.

The present invention concerns the use of omega-3 fatty acids for the treatment of hypercholesterolaemia caused by antiretroviral treatment of patients infected with Human Immunodeficiency Virus (HIV).

The present invention also concerns a product containing at least one omega-3 fatty acid and at least one antiretroviral agent as combination product for simultaneous, separate or sequential use to treat hypercholesterolaemia caused by antiretroviral treatment in HIV-infected patients.

Although antiretroviral treatments currently allow the mortality rate of HIV-infected patients to be considerably reduced, they very often cause various metabolic disorders in these patients, in particular lipid metabolism disorders such as hyperlipidaemia and especially hypercholesterolaemia.

This secondary hyperlipidaemia appears to be due to liver activation caused by the protease inhibitors used for antiretroviral treatment.

Lipid metabolism disorders may, in time, lead to serious cardiovascular complications. They may therefore seriously reduce the life expectancy of patients receiving an antiretroviral treatment if the condition is uncontrolled.

The diagnosis of hyperlipidaemia is based on a blood lipid test particularly including measurement of the total cholesterol (TC) plasma concentration and total triglyceride (TG) plasma concentration.

Cholesterol and triglycerides are not transported as such in the blood plasma. These highly hydrophobic substances require a special transport system allowing their solubilisation.

Triglyceride-rich lipoproteins (TRLs) are of hepatic or intestinal origin. These lipoproteins contain both triglycerides and cholesterol (free or esterified). The progressive loss of triglycerides, under the effect of the lipoprotein lipase and hepatic lipase, leads to the formation of smaller, denser lipoproteins relatively enriched with cholesterol (Intermediate Density Lipoproteins—IDLs; and Low Density Lipoproteins—LDLs).

Fragments of the phospholipid-protein envelope of these TRLs, associated with free cholesterol, detach themselves giving rise to high density particles: High Density Lipoproteins—HDLs).

Therefore, the metabolic outcome of TRLs naturally leads to an increase in the plasma availability of LDLs and HDLs. Under physiological conditions, the uptake of these lipoprotein particles allows a stable level to be maintained of the two cholesterol-rich classes (LDLs and HDLs).

In the event of excess VLDLs (Very Low Density Lipoproteins: TRLs of hepatic origin) the rapid conversion by the lipoprotein lipase leads to an, at least transitional, increase in these cholesterol-rich fractions, LDLs and HDLs. The reverse relationship between levels of HDL-cholesterol and plasma triglycerides has been evidenced for many years.

Reducing LDL levels has long been the major objective of treatments against hypercholesterolaemia. However, it would appear that other lipoproteins play an important role in the development of cardiovascular disease.

Recent studies have shown that the non-HDL fraction of circulating lipoproteins (which contains LDLs, IDLs and VLDLs) represents the major risk factor for some cardiovascular diseases, such as myocardial infarction or atherosclerosis.

Omega-3 fatty acids have well established hypotriglyceridemic properties. The mechanisms of action of this effect were partly specified in the work reported by the Brown and Goldstein team. These substances regulate the expression of the genes involved in the liver synthesis of sterols and fatty acids.

Having regard to the transport system for lipids circulating in the plasma, it is impossible to modify one of the basic constituents (triglycerides, cholesterol, phospholipids, etc.) without modifying the other parameters.

While the effect of omega-3 fatty acids on triglycerides has been well established, the beneficial effect of these same substances on the metabolism of circulating cholesterol has, up until now, never been proven. Most often, in the general hyperlipidemic population, the omega-3 fatty acids tend to cause the cholesterol level to be increased, in particular its fraction bound to the atherogenic LDLs, especially during short term treatments.

Unexpectedly, the Applicant has discovered that the administering of omega-3 fatty acids to HIV-infected patients receiving an antiretroviral treatment allows their plasma cholesterol level to be maintained, or its increase to be limited. More precisely, the administering of omega-3 fatty acids can maintain or limit the non-HDL fraction of circulating lipoproteins.

Therefore in HIV-infected patients receiving an antiretroviral treatment, omega-3 fatty acids can be used to prevent or treat hypercholesterolaemia caused by this antiretroviral treatment, in particular when diet, physical exercise or a change in antiretroviral treatment prove to have no beneficial effect on the cholesterol metabolism.

The subject-matter of the present invention is the use of omega-3 fatty acid(s) or a marine and/or plant source of omega-3 fatty acid(s) to prepare a medicament intended to treat hypercholesterolaemia caused by an antiretroviral treatment in patients infected with Human Immunodeficiency Virus (HIV), by limiting the non-HDL fraction of circulating lipoproteins.

By treatment of hypercholesterolaemia caused by an antiretroviral treatment, is meant the preventive or curative treatment of this hypercholesterolaemia.

By preventive treatment of hypercholesterolaemia is meant a treatment intended to prevent the onset of hypercholesterolaemia, i.e. to maintain the total cholesterol plasma level at a level of around 2 g/l or less.

By curative treatment of hypercholesterolaemia is meant a treatment intended to reduce the total cholesterol plasma level to a level of approximately 2 g/l or less.

One particular object of the invention is the preparation of a medicament intended for the preventive treatment of hypercholesterolaemia caused by an antiretroviral treatment in patients infected with Human Immunodeficiency Virus (HIV).

Omega-3 fatty acids are polyunsaturated fatty acids which have a double bond located at three carbon atoms from the methyl end of the molecule. They take part in the production of highly unsaturated fatty acids and 3-series eicosanoids. These substances play a central role at the cell membranes and take part in numerous biochemical processes of the body: regulating arterial pressure, elasticity of vessels, immune and anti-inflammatory reactions, blood platelet aggregation, etc.

Within this family, solely alpha-linolenic acid (ALA) is said to be “essential”. The other omega-3 fatty acids can be produced by the body from ALA. It is particularly found in linseed and linseed oil, in hemp seed and hemp oil, and in canola and soybean oil.

Eicosapentaenoic acid (EPA) is a fatty acid synthesized from ALA, but can also be found directly in some foods particularly in some oily fish. EPA converts to series-3 eicosanoids which inter alia contribute towards protecting the heart and arteries and have anti-inflammatory and anti-allergic effects.

Docosahexaenoic acid (DHA) is another derivative of the conversion of omega-3 acids. It is also found in marine products, in particular in some oily fish. It plays a fundamental role in the development of the brain and retina, and in the formation of spermatozoid motility.

Under the present invention, the omega-3 fatty acids are advantageously chosen from among alpha-linolenic acid (ALA), eicosapentaenoic acid (EPA), docosahexaenoic acid (DHA), docosapentaenoic acid (DPA) and stearidonic acid, and their mixtures.

The plant source of omega-3 fatty acid(s) is advantageously chosen from among linseed and linseed oil, hemp seed and hemp seed oil, pumpkin seed and pumpkin seed oil, soybean oil, canola oil and/or blackcurrant pip oil.

The marine source of omega-3 fatty acid(s) is advantageously chosen from among oils extracted from fish meat such as mackerel, salmon, herring, sardines, tuna or trout, krill oil and/or seal oil.

The omega-3 fatty acids or their marine and/or plant source may be administered by oral route, in particular as food supplement, or via parenteral route (by injection).

Advantageously, the taking of omega-3 fatty acids by patients is combined with a food diet or physical exercise intended to reduce cholesterol plasma levels.

Preferably, a natural fish oil is used e.g. containing a concentration of 30% omega-3 fatty acids, such as the oil marketed in France under the name MAXEPA® in the form of capsules for oral administering in which 1 g of oil contains 180 mg EPA and 120 mg DHA.

Under the present invention, by antiretroviral treatment is meant a treatment against HIV infection which uses reverse transcriptase inhibitors (RTIs) and/or protease inhibitors (PIs).

RTIs act in the cell to hinder the action of a viral enzyme, the reverse transcriptase, and thereby prevent the transcription of viral RNA into viral DNA which paralyzes the DNA of the host cell.

PIs act at another stage of HIV reproduction by attacking the activity of the protease, a viral enzyme permitting the maturing of new viruses created by the infected cell. Through the action of protease inhibitors (which are up to 1000 times more powerful than RTI inhibitors), the cell produces immature virions incapable of infecting new cells.

The antiretroviral treatment of the present invention may use one or more reverse transcriptase inhibitors (RTIs) optionally in association with one or more protease inhibitors (PIs).

Therefore, the antiretroviral treatment according to the present invention may be a biotherapy i.e. using an association of two RTIs, or a tritherapy i.e. using an association of one PI with two RTIs, or anti-retroviral multi-therapy i.e. an association of one or more PIs with one or more RTIs, conventionally an association of 4 products.

Advantageously, the omega-3 fatty acids are administered to the proportion of 1 to 3 g/d, preferably around 2 g/d.

Use of the omega-3 fatty acids according to the invention proves to be particularly effective in HIV-infected patients receiving an antiretroviral treatment and suffering from hyperlipidaemia with predominant hypertriglyceridaemia caused by this antiretroviral treatment.

A further subject of the present invention is a product containing at least one omega-3 fatty acid or a marine and/or plant source of omega-3 fatty acid and at least one antiretroviral agent, as combination product for simultaneous, separate or sequential use to treat hypercholesterolaemia caused by an anti-retroviral treatment in patients infected with Human Immunodeficiency Virus (HIV), by limiting the non-HDL fraction of circulating lipoproteins.

Advantageously, the omega-3 fatty acid is chosen from among alpha-linolenic acid (ALA), eicosapentaenoic acid (EPA), docosahexaenoic acid (DHA), docosapentaenoic acid (DPA) and stearidonic acid, and their mixtures.

Advantageously, the plant source of omega-3 fatty acid(s) is advantageously chosen from among linseed and linseed oil, hemp seed and hemp seed oil, pumpkin seed and pumpkin seed oil, soybean oil, canola oil and/or blackcurrant pip oil.

Advantageously, the marine source of omega-3 fatty acid(s) is chosen from among oils extracted from fish meat such as mackerel, salmon, herring, sardines, tuna or trout, krill oil and/or seal oil.

According to the present invention, the anti-retroviral agent is chosen in particular from among reverse transcriptase inhibitors (RTIs) and protease inhibitors (PIs) and their associations.

The RTIs can be chosen from among zidovudine, didanosine, zalcitabine, lamiduvine, neviparine, delacirdine and efavirenz and their associations, and the PIs can be chosen from among ritonavir, indinavir, saquinavir and sulfinavir and their associations.

As indicated above the omega-3 fatty acid, or an animal source and/or plant source thereof, and the antiretroviral agent contained in the combination product of the invention may be used simultaneously, separately or sequential over time.

Simultaneously refers to co-administering. In this case, the two essential components may be mixed prior to administering or may be administered at the same time. It is also possible to administer them separately i.e. one after the other irrespective which component of the combination product is administered first. Finally, administering may be sequential over time or may intermittent when it is stopped and resumed at regular or irregular intervals. The routes and sites chosen to administer the two components may be different.

As indicated above omega-3 fatty acids, or their animal and/or plant source, are generally administered via oral route, in particular as food supplement, or via parenteral route.

Preferably, a natural fish oil is used in the combination product, e.g. having a 30% concentration of omega-3 fatty acid such as the product marketed in France under the name MAXEPA®.

The anti-retroviral agents are generally given via oral route.

Preferably, the omega-3 fatty acids are taken after the start of the antiretroviral treatment if treatment targets hypercholesterolaemia, or before the start of antiretroviral treatment if prevention of hypercholesterolaemia is targeted.

Advantageously, the combination product according to the invention contains an efficient daily dose of omega-3 fatty acids of between 1 and 3 g omega-3 fatty acids. The quantity of omega-3 fatty acids contained in the combination product varies in relation to different parameters, e.g. the individual to be treated or the type of treatment, namely if it is preventive or curative.

Regarding the antiretroviral agents, the adequate quantity is the quantity usually used in the treatment of HIV infections.

The present invention is illustrated by the following examples:

### EXAMPLES

A) Studies on a mouse model whose diet was enriched with omega-3 fatty acids showed:
accelerated turnover of High Density Lipoproteins (HDLs),
increased cholesterol efflux from body peripheral cells,
increased expression of the SR-B1 receptors participating in the liver uptake of HDLs,
increased expression of the gene regulating the expression of 7-hydroxylase and sterol-15α-hydroxylase responsible for cholesterol degradation,
increased faecal elimination of sterols, which is increased twofold in treated animals.

All these elements lead to cholesterol depletion in the bodies of treated mice, and take part in the anti-atheromatic property of omega-3 fatty acids.

B) A clinical trial in HIV-patients treated with antiretroviral polytherapy and suffering from predominant hypertriglyceridaemia, evidenced the reality of the regulatory process on circulating cholesterol levels by omega-3 fatty acids, the treated group maintaining its cholesterol level while the placebo group showed increasing figures despite the diet followed throughout the trial.

The daily intake of 6 grams of oil with a 30% concentration of omega-3 fatty acids (EPA and DHA) by HIV-infected patients treated with antiretroviral polytherapy, and suffering from hyperlipidaemia with predominant hypertriglyceridaemia, brought a reduction in total tryglyceridaemia, after 8 weeks' treatment, associated with stabilisation of cholesterol levels compared with the placebo. This stabilisation of cholesterol levels concerns the non-HDL fraction of circulating lipoproteins, as evidenced by the slight increase in HDL-Cholesterol during this trial in the group treated with the active ingredient.

Therefore omega-3 fatty acids have a positive influence on the metabolism of plasma cholesterol within the specific context of HIV-infected patients treated with antiretroviral polytherapy who suffer from hyperlipidaemia with predominant hypertriglyceridaemia.

1-15. (canceled)

16. Method for treating hypercholesterolaemia caused by an antiretroviral treatment in a patient infected with Human Immunodeficiency Virus (HIV), by limiting the non-HDL fraction of circulating lipoproteins, comprising administering to said patient an effective amount of omega-3 fatty acid(s), or a marine and/or plant source of omega-3 fatty acid(s).

17. Method according to claim 16, for the preventive treatment of hypercholesterolaemia caused by an antiretroviral treatment in patients infected with Human Immunodeficiency Virus (HIV).

18. Method according to claim 16, wherein the omega-3 fatty acids are selected from the group consisting of alpha-linolenic acid (ALA), eicosapentaenoic acid (EPA), docosahexaenoic acid (DHA), docosapentaenoic acid (DPA) and stearidonic acid, and their mixtures.

19. Method according to claim 16, wherein the plant source of omega-3 fatty acid(s) is selected from the group consisting of linseed and linseed oil, hemp seed and hemp seed oil, pumpkin seed and pumpkin seed oil, soybean oil, canola oil and blackcurrant pip oil.

20. Method according to claim 16, wherein the marine source of omega-3 fatty acid(s) is oil extracted from fish meat.

21. Method according to claim 16, wherein the HIV-infected patient is receiving an antiretroviral treatment and suffers from hyperlipidaemia with predominant hypertriglyceridaemia caused by this antiretroviral treatment.

22. Method according to claim 21, wherein the antiretroviral treatment is an antiretroviral biotherapy, tritherapy or multitherapy.

23. Method according to claim 21, wherein the antiretroviral treatment uses one or more reverse transcriptase inhibitors (RTIs) optionally in association with one or more protease inhibitors (PIs).

24. Method according to claim 16, wherein said omega-3 fatty acid(s) or a marine source and/or plant source of omega-3 fatty acid(s) are administered with at least one antiretroviral agent simultaneously, separately or sequentially.

25. Method according to claim 24, wherein the fatty acid(s) are selected from the group consisting of alpha-linolenic acid (ALA), eicosapentaenoic acid and docosahexaenoic acid (DHA), docosapentaenoic acid (DPA) and stearidonic acid, and their mixtures.

26. Method according to claim 24, wherein the plant source of omega-3 fatty acid(s) is selected from the group consisting of linseed and linseed oil, hemp seed and hemp seed oil, pumpkin seed and pumpkin seed oil, soybean oil, canola oil and blackcurrant pip oil.

27. Method according to claim 24, wherein the marine source of omega-3 fatty acid(s) is oil extracted from fish meat.

28. Method according to claim 24, wherein the antiretroviral agent is selected from the group consisting of reverse transcriptase inhibitors (RTIs) and protease inhibitors (PIs) and their associations.

29. Method according to claim 28, wherein the reverse transcriptase inhibitors are selected from the group consisting of zidovudine, didanosine, zalcitabine, lamiduvine, neviparine, delavirdine and efavirenz, and their associations.

30. Method according to claim 28, wherein the protease inhibitors are selected from the group consisting of ritonavir, indinavir, saquinavir and sulfinavir, and their associations.

31. Method according to claim 20, wherein the fish meat is selected from the group consisting of mackerel, salmon, herring, sardines, tuna or trout, krill oil and seal oil.

32. Method according to claim 27, wherein the fish meat is selected from the group consisting of mackerel, salmon, herring, sardines, tuna or trout, krill oil and seal oil.
